(19) United States
(12) Patent Application Publication
(10) Pub. No.: US 2013/0245347 A1
(43) Pub. Date: Sep. 19, 2013

(54) DEVICE AND METHOD FOR ARYL-ALKYL COUPLING USING DECARBOXYLATION

(71) Applicant: CERAMATEC, INC., Salt Lake City, UT (US)

(72) Inventors: James Mosby, Salt Lake City, UT (US); Patrick McGuire, Salt Lake City, UT (US); Sai Bhavaraju, West Jordan, UT (US); Mukund Karanjikar, West Valley City, UT (US)

(73) Assignee: CERAMATEC, INC., Salt Lake City, UT (US)

(21) Appl. No.: 13/834,569

(22) Filed: Mar. 15, 2013

Related U.S. Application Data
(63) Continuation-in-part of application No. 12/840,401, filed on Jul. 21, 2010.
Application No. 12/840,401 is a continuation-in-part of application No. 12/840,913, filed on Jul. 21, 2010.
Application No. 12/840,913 is a continuation-in-part of application No. 12/840,508, filed on Jul. 21, 2010.
Application No. 12/840,508 is a continuation-in-part of application No. 13/612,192, filed on Sep. 12, 2012.
Application No. 13/612,192 is a continuation-in-part of application No. 13/092,685, filed on Apr. 22, 2011.
Application No. 13/092,685 is a continuation-in-part of application No. 13/790,744, filed on Mar. 8, 2013.
(60) Provisional application No. 61/258,557, filed on Nov. 5, 2009.
(60) Provisional application No. 61/228,078, filed on Jul. 23, 2009.
(60) Provisional application No. 61/260,961, filed on Nov. 13, 2009.

(51) Int. Cl. C25B 3/10; C25B 9/08; C07C 6/04

(52) U.S. Cl. CPC C25B 3/10; C07C 6/04; C25B 9/08
USPC 585/320; 585/310; 585/323; 204/252; 204/242

(57) ABSTRACT

A method for alkylating aromatic compounds is described using an electrochemical decarboxylation process. This process produces aryl-alkyl compounds that have properties useful in Group V lubricants (and other products) from abundant and economical carboxylic acids. The process presented here is also advantageous as it is conducted at moderate temperatures and conditions, without the need of a catalyst. The electrochemical decarboxylation has only H₂ and CO₂ as its by-products, as opposed to halide by-products.

### RELATED APPLICATIONS

This application is a continuation-in-part of U.S. patent application Ser. No. 12/840,401, filed Jul. 21, 2010, which in turn, claims the benefit of U.S. Provisional Patent Applications Nos. 61/258,557 filed Nov. 5, 2009, 61/228,078 filed Jul. 23, 2009 and 61/260,961 filed Nov. 13, 2009. This application is also a continuation-in-part of U.S. patent application Ser. No. 12/840,913, filed Jul. 21, 2010. This application is also a continuation-in-part of U.S. patent application Ser. No. 12/840,508, filed Jul. 21, 2010. This application is also a continuation-in-part of U.S. patent application Ser. No. 13/612,192, filed Sep. 12, 2012. This application is also a continuation-in-part of U.S. patent application Ser. No. 13/092,685, filed Apr. 22, 2011. This application is also a continuation-in-part of U.S. patent application Ser. No. 13/790,744, filed Mar. 8, 2013.

All of the above-recited provisional and non-provisional applications are expressly incorporated herein by reference.

### TECHNICAL FIELD

The present disclosure relates to a method of producing hydrocarbon materials such as lubricants and other useful products. More specifically, the present disclosure provides an electrochemical decarboxylation process in which aryl and alkyl groups are coupled together to form useful products.

### BACKGROUND

The above-recited patent applications teach methods of forming hydrocarbons and other molecules using an electrochemical decarboxylation process (“EDP”). The reader is presumed to be familiar with the disclosure and content of these prior applications.

Industrial lubricants are important to many processes and applications and are commonly classified into the following five groups. Group I lubricants contain less than 90% saturated hydrocarbons and/or have more than 0.03% sulfur present and are manufactured using solvent extraction and hydro-finishing processes. Group II lubricants contain more than 90% saturated hydrocarbons and/or have less than 0.03% sulfur present and are manufactured using hydrocracking and solvent or catalytic dewaxing processes. Group III lubricants have more than 90% saturated hydrocarbons and less than 0.03% sulfur present and are manufactured using special processes such as isohydromerization. Group IV lubricants are lubricants based on polyalphaolefins. Group V lubricants are lubricants that do not fall into any of the other groups such as diesters, polyesters, alkylated naphthalenes, and alkylated benzenes. Each classification of lubricants find use in different applications based on cost and application requirements. The different types of lubricants within Group V provide superior performance compared to the other lubricants classes in regards to a specific property, for example corrosion-resistance.

One class of compounds that make up the Group V lubricants are alkylated aromatic (AR) compounds. There are two main types of AR compounds. The first group constitutes compounds in which benzene makes up the aromatic component of the compound. The second group constitutes compounds in which naphthalene makes up the aromatic component of the compound. The non-aromatic components of AR compounds found in Group V lubricants are usually long chain hydrocarbons with different degrees of saturation. The properties of AR compounds are determined by the type of aliphatic and aromatic components that are coupled and also by the number of alkyl components attached to the aromatic center. The properties of alkyl-aryl compounds can be easily adjusted by changing the length and/or number of the alkyl components attached to the aromatic component. Thus, one could tailor the properties of the lubricant for specific applications by altering the aliphatic component and by varying the degree that the aromatic group is alkylated.

It would be advantageous to find a manufacturing method to produce alkylated aromatic compounds that are useful as Group V lubricants and other products. It would further be advantageous to find manufacturing methods that use inexpensive starting materials and/or economically sustainable processes, for example processes that do not rely on expensive catalysis, high temperatures and/or high pressures. Conventional methods for preparing AR compounds for Group V lubricants are based the Friedel-Crafts alkylation which requires a catalyst and high temperatures to perform the alkylation. Also, conventional methods require the use of organo- or metallic halides, thereby producing a halide waste stream as the reaction by-product. It is the aim of the present disclosure to synthesize alkylated aromatics suitable for Group V lubricants from carboxylic acids using mild conditions and temperatures, with the only by-products being CO₂ and H₂. It would also be an improvement to provide a method of producing alkylated aromatic compounds in which the degree of alkylation of the aromatic ring may be tailored and controlled. Such methods are disclosed herein.

Carboxylic acids are a popular starting material for synthesizing industrially important compounds because such acids are economically and environmentally friendly. One application using carboxylic acids involves using the acids as alternatives to organohalides in the Heck reaction for the formation of carbon-carbon double bonds. (The Heck reaction is a catalytic reaction between an organohalide with an alkene and a base to form a substituted alkene.) Replacing the organohalide of the Heck reaction with a carboxylic acid is more environmentally friendly because CO₂ and H₂ are the only by-products formed as opposed to halide by-products. Carboxylic acids are also being investigated as substrates for cross-coupling reactions where the carboxylic acid can act as either the nucleophilic or electrophilic coupling partner. This is advantageous as there are a large number of carboxylic acids available commercially which are more economical than conventionally used organohalides and/or organometallic reagents. (These reactions are described by in the article by W. I. Dzik, P. P Lange, L. J. GooBen, Chemical Science, 2012, 3, 2671). While the reactions describe above benefit from the availability and low cost of carboxylic acids, they still require a catalyst and high temperatures to promote the transformations. It would be beneficial to find methods of carbon-carbon coupling using carboxylic acid substrates that do not require the use of catalyst and can be performed at moderate temperatures and reaction conditions. Such reactions are disclosed herein.

### SUMMARY

The present embodiments relate to methods to alkylate aromatic compounds by performing alkyl-aryl coupling via EDP. These alkyl-aryl coupling reactions can be used to prepare compounds suitable for many applications, including, for example, compounds classified as Group V lubricants. The EDP converts alkali salts of carboxylic acids to radicals which can then go through radical-radical coupling. This process is known as a modified Kolbe electrolysis. When performed in the presence of a single carboxylic acid, this process leads to homocoupling of radical species. As described in this application, the electrolysis can also be performed in the presence of more than one carboxylic acid which leads to heterocoupling of radical species. The heterocoupling can couple radicals from different carboxylic acids, and as disclosed herein, can couple radicals containing alkyl and aromatic functional groups. Such an alkyl-aryl couple provides an inexpensive method to alkylate aromatic compounds.

The present embodiments may further involve methods to produce alkylated aromatic compounds that have properties that are desired for Group V lubricants. The methods may involve the oxidation of carboxylic acids using an electrochemical cell. The electrolysis may be performed in the presence of at least one alkyl carboxylate salt and at least one aryl functionalized compound. The aryl compound may itself be a carboxylic acid or an alkali metal salt of a carboxylic acid or, in other embodiments, may be an aromatic compound that interacts with the alkyl through double bonds on its aromatic ring. Thus, the electrolysis creates radicals which may undergo heterocoupling that generates alkylated aromatic compounds. Alternatively, the electrolysis products may be involved in electrophilic substitution reactions which may also generate alkylated aromatic compounds.

Prior to the electrolysis driven alkylation described herein, the carboxylic acids may be first converted to alkali metal salts via conventional saponification procedures. This saponification reaction may involve reacting the carboxylic acids with an alkali metal base (MOH) at an elevated temperature (or at some other temperature). Some non-limiting examples of alkali metal bases are lithium hydroxide, sodium hydroxide, potassium hydroxide, alkoxides, etc. The generic neutralization can be represented as follows:

RCO₂H+MOH→RCO₂M+H₂O

In one embodiment, this saponification reaction is carried out in a solvent with an alkoxide present, and the reaction forms an alkali carboxylate which precipitates out of solution. In such an embodiment, the alkali carboxylate salt can be easily isolated to prepare the anolyte needed for the decarboxylation process.

Alkali carboxylates may then be electrochemically decarboxylated leading to the formation of radical coupling products. Due to the presence of multiple carboxylate ions produced by decarboxylation, both homocoupling and heterocoupling products are obtained. This radical coupling process may be performed using a two compartment electrochemical cell which is made using a NaSelect® membrane that is commercially available from Ceramatec, Inc., of Salt Lake City, Utah. The electrolysis in the anolyte compartment follows the modified Kolbe electrolysis as shown in the reaction below.

2R₁CO₂M→R₁—R₁+2CO₂+2e⁻+2M⁺

2R₂CO₂M→R₂—R₂+2CO₂+2e⁻+2M⁺

R₁CO₂M+R₂CO₂M→R₁—R₂+2CO₂+2e⁻+2M+

In one embodiment, the R₁ refers to an aliphatic carboxylate salt and R₂ refers to an aromatic carboxylate salt. While all three of the reactions above can occur during the electrolysis, preferably the electrolysis is performed in a manner that favors the formation of heterocoupling product (R₁—R₂) over the homocoupling products (R₁—R₁ and R₂—R₂).

In another embodiment, the electrolysis is performed on an aliphatic carboxylate salt in the presence an aromatic compound, for example, benzene. During this reaction, a substitution reaction occurs that couples the aliphatic group to the aromatic ring (benzene):

R₁CO₂M+C₆H₆→C₆H₅—R₁+CO₂+2e+H⁺+M⁺

In the case shown above, the decarboxylation of the aliphatic carboxylate forms an electrophile which then can undergo electrophilic substitution on an aromatic ring. The electrophile can be in the form of a radical or carbocation. The latter is generated by a two electron oxidation during the decarboxylation step instead of a one electron oxidation.

### BRIEF DESCRIPTION OF THE DRAWINGS

FIG. 1 shows a schematic drawing of an electrochemical cell that may be used to decarboxylate alkali metal salts of carboxylic acids;

FIG. 2 is a plot of the potential and current density of the electrochemical decarboxylation of sodium oleate and sodium benzoate;

FIG. 3 shows a gas chromatogram of the products obtained from the electrochemical decarboxylation of sodium oleate and sodium benzoate;

FIG. 4 is a plot of the potential and current density of the electrochemical decarboxylation of sodium naphthenate;

FIG. 5 shows a gas chromatogram of the products obtained from the electrochemical decarboxylation of sodium naphthenate;

FIG. 6 is a plot of the potential and current density of the electrochemical decarboxylation of sodium naphthenate and sodium naphthoate;

FIG. 7 shows a gas chromatogram of the products obtained from the electrochemical decarboxylation of sodium naphthenate and sodium naphthoate;

FIG. 8 is a plot of the potential and current density of the electrochemical decarboxylation of sodium naphthenate and sodium naphthoate in a mixture of polar and non-polar organic solvents; and

FIG. 9 shows a Simulated Distillation (SimDist) of the products obtained from the electrochemical decarboxylation of sodium naphthenate and sodium naphthoate in a mixture of polar and non-polar organic solvents.

### DETAILED DESCRIPTION

Some terms and their definitions that will be used throughout this description are provided. “Lubricant,” as used herein, refers to substances that are used to reduce the friction between moving surfaces. “Alkane” and/or “aliphatic” are defined as a saturated hydrocarbon, or mostly saturated hydrocarbon, and will be used interchangeably throughout the disclosure. “Alkyl” is defined as a hydrocarbon alkane that is missing one bond, such as from the removal of a carboxyl group. “Aryl” is defined as a compound that contains at least one aromatic ring as a functional group or substituent. “Aromatic” is defined as a cyclic compound with alternating double and single bonds between carbon atoms forming a ring with a conjugated pie system. “Carboxylic acid” is a compound with the general formula RCO₂H, where the “R” can represent saturated or unsaturated hydrocarbon chains. “Naphthenic acid” refers to a mixture of carboxylic acids with cyclopentyl and cyclohexyl groups with a carbon backbone between 9 and 20 carbons, and a molecular weight of between 120 and 700 amu. “Decarboxylation” herein refers to the process of removing CO₂ from a compound, specifically from a carboxylic acid or anion.

A type of electrolytic decarboxylation process which is concerned with making surfactants from aryl-alkyl coupling is described in U.S. Patent Application Publication No. 2011/0226633, which publication is expressly incorporated herein by reference.

The present embodiments teach a method to produce alkylated aromatics (AR) products which may, for example, be used as components in Group V lubricants. The properties of the formed AR products depend on the structure of both the alkyl and aryl components as well as the number of alkyl components that are coupled to a single aryl component. Common methods of preparing AR compounds are based on the Friedel-Crafts alkylation which uses a catalyst to alkylate aromatic compounds. Such a process can lead to the formation of monoalkylaromatics (MAR), dialkylaromatics (DAR) and polyalkaromatics (PAR). Because the properties of the MAR, DAR, and PAR may differ significantly from each other, a material with the desired properties is obtained by separating the different compounds through distillation and/or blending. One advantage of the present embodiments is that it provides control over the number of alkyl chains that attach to the aromatic component, and thus provides control over the properties of the synthesized compounds.

There are a large number of inexpensive carboxylic acid substrates that are available to use as the alkyl component of the alkyl-aryl coupling product. These carboxylic acid substrates can be coupled to a large number of possible aromatic compounds. The abundance of inexpensive substrates enhances the ability to control and fine-tune the properties of the synthesized AR compound to match the specific needs of the lubricant application (or any other desired application). The length of the alkyl group may affect the physical properties of the material, such as pour point, viscosity index, and flash point. The substitution on the aromatic system may increase the pour point, the viscosity index, and the flash point. The aryl component of the alkyl-aryl compound may affect the thermo-oxidative stability of the formed compound (because the electron-rich aromatic portion of the molecule can scavenge radicals and disrupt oxidation processes).

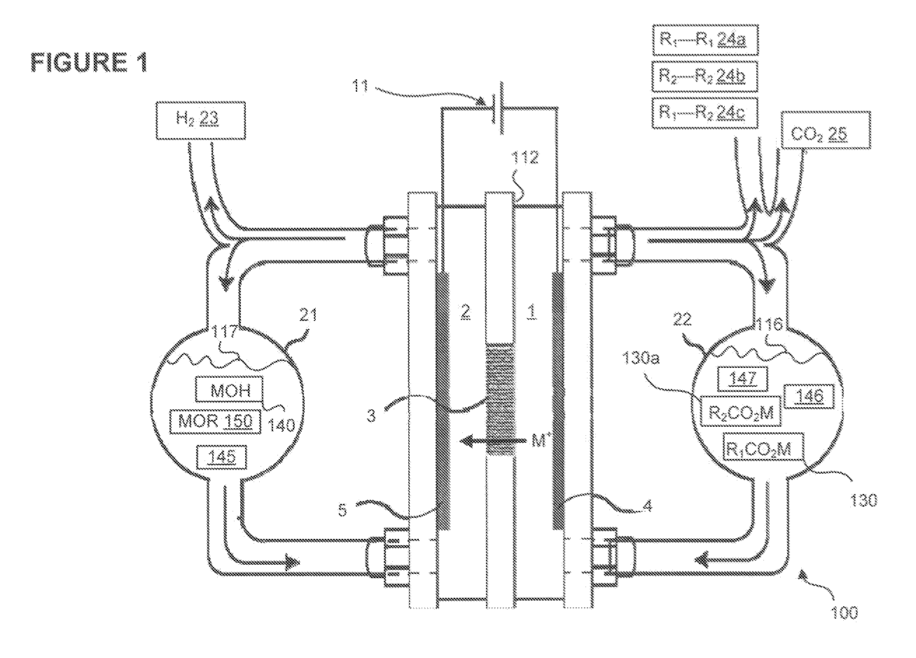

Feasible and economical industrial processes for coupling alkyl-aryl compounds involve the use of catalysts and/or high temperatures. The present embodiments describe processes for coupling alkyl-aryl compounds that may not require catalysts and/or high temperatures. The present processes may use an electrolytic cell 100 schematically represented in FIG. 1. The cell 100 may have two compartments, namely an anode chamber 1 and a cathode chamber 2. The chambers 1, 2 may be separated by an alkali metal ion conductive membrane 3. This membrane 3 may be, for example, a NaSelect® membrane. Anolyte 116 may be fed into the anode chamber 1 (which may also be referred to as the “anode compartment”). During electrolysis, components of the anolyte 116 are oxidized at the surface of an anode 4, causing decarboxylation of the carboxyl functional group to form radicals and CO₂. Then, according to one embodiment, these radicals can react to form a long chain aliphatic compound, or, according to another embodiment, they can react to form a polycyclic aromatic compound, and in yet still another embodiment, the radicals can react to form an aryl-alkyl compound. Depending on the conditions used for the electrolysis and the relative activation energy of the decarboxylation, the radicals could react leading to all embodiments described above, or only according to one of the above-recited embodiments. The anode 4 is housed, either fully or partially, within the anode chamber 1.

On the other side of the cell 100, the reduction of a catholyte 117 is occurring. This reduction occurs in a cathode compartment 2 (which may also be referred to as the “cathode chamber”). A cathode 5 is housed, either fully or partially, in the cathode chamber 2. To maintain charge balance, a positive ion must transfer from the anode 4 to the cathode 5, and in the case when the anolyte 116 and catholyte 117 are separated, there needs to be a path for the positive ions to transfer between the compartments 1, 2. In one embodiment, the ion conducting membrane 3 selectively transfers alkali ions (M+), including but not limited to the ions of, sodium, lithium, and potassium, from the anolyte 116 to the catholyte 117 under the influence of an applied electrical field.

In one embodiment, the ion conductive membrane 3 is between 10 and 5000 microns thick, or more preferably, the membrane is between 100 and 1000 microns thick, or even more preferably, the membrane is between 200 and 700 microns thick. In another embodiment, the membrane 3 is in the form of disk with diameters between 0.25 and 25 cm, even more preferably, the diameter is between 1.27 and 12.7 cm, or most preferably, between 2.54 and 7.62 cm. The membrane 3 may be assembled in a scaffold 112. In another embodiment, the membrane 3 is in the form of a cylinder with a diameter between 0.25 and 25 cm, even more preferably, between 1.27 and 12.7 cm, or most preferably, between 2.54 and 7.62 cm.

Thus, in one embodiment the electrochemical cell 100 can be in a parallel plate configuration which uses flat membranes, for example, as shown in FIG. 1. In another embodiment, the electrochemical cell 100 is in a tubular configuration which uses tubular electrodes and membranes. It should be clear to one skilled in the art that the cell configurations listed above both have advantages and disadvantages which would lead to one being chosen over the other depending on the requirements of the specific carboxylic salt being decarboxylated. The process described herein can be applied in a variety of cell designs (including those described above and other cell configurations).

The anode 4 can comprise any suitable material that allows oxidation reactions to occur in the anode compartment 1 when an electrical field 11 is applied between the anode 4 and cathode 5. Some non-limiting examples of anode materials include, but are not limited to, platinum, titanium, nickel, cobalt, iron, stainless steel, lead dioxide, metal alloys, combination thereof, and other known or novel anode materials. In one embodiment, the anode 4 may be comprised of iron-nickel alloys such as KOVAR® or INVAR®. In other embodiments, the anode 4 may be comprised of carbon based electrodes such as boron doped diamond, glassy carbon, and synthetic carbon. Additionally, in some embodiments, the anode 4 may comprise a dimensionally stable anode (DSA), which may include, but is not limited to, rhenium dioxide and tantalum pentoxide on a titanium substrate.

The cathode 5 may also be fabricated of any suitable cathode material that allows the reduction of water or methanol to produce hydroxide or methoxide ions and hydrogen gas. The cathode 5 may be comprised of the materials used for the anode 4. Alternatively, the cathode 5 may comprise materials different from that used as the anode 4. Some non-limiting examples of suitable cathode materials include, without limitation, nickel, stainless steel, graphite, and any other suitable cathode material that is known or novel.

In one embodiment, the electrodes 4, 5 have a smooth morphology such as a foil or thin film. In another embodiment, the anode 4 and cathode 5 have a high surface area morphology, for example, but not limited to, a foam, grit, or other porous structure. In some embodiments, the anode 4 and cathode 5 have the same morphology, while in other embodiments, the electrodes 4, 5 have different morphologies.

In the embodiment of FIG. 1, the anolyte 116 is housed in a reservoir 22 and may be fed into the anode compartment 1. The catholyte 117 may likewise be housed in a reservoir 21 and fed into the cathode compartment 2. The cathode compartment 2 may be separated from the anode compartment 1 by the ion conductive membrane 3. The anolyte 116 may comprise a solvent 146 and alkali salts of carboxylic acids 130/130a. (The carboxylic acid itself may also be in the anolyte 116, as desired.) The anolyte 116 may comprise a mixture of alkali salts of carboxylic acids, namely R₁CO₂M 130 and R₂CO₂M 130a. At least one of the acids or salts may be an alkyl carboxylic acid, and another of the salts may be an aryl carboxylic acid. In another embodiment, the anolyte 116 may comprise a compound with an aryl group (such as benzene) that is used as the solvent 146. In this system, the anolyte 116 may only contain one alkali salt of a carboxylic acid 130, which may be an alkali salt of carboxylic acids that is aliphatic in nature.

The catholyte 117 may comprise a solvent 145. The solvent 145 may or may not be the same as the solvent 146 in the anode compartment 2. One of the advantages of using a cell divided by a membrane 3, is that the solvents used on each side do not have to be the same; rather, the solvent used on each side of the membrane 3 may be tailored for the particular reaction occurring in each separate compartment. The catholyte 117 may be a conductive solution that may include an alkali metal hydroxide 140 and/or an alkali metal methoxide 150. Thus, the anolyte and catholyte solvents 146, 145 may be separately selected specifically for the reactions that occur in each compartment and/or the solubility of the chemicals required for the specific reactions. This permits a cell designer to construct an inexpensive catholyte 117 which may have different properties than the anolyte 116, if desired. For example, the catholyte 117 may be designed such that it has high ionic conductivity.

In one embodiment, the anolyte solution 116 may comprise a solvent 146 that is a polar organic solvent. Some non-limiting examples of suitable polar organic solvents include, without limitation, methanol, ethanol, isopropanol, n-propanol, acetone, acetonitrile, acrylonitrile and glycerol. In other embodiments, the solvent 146 may comprise an aromatic solvent. Some non-limiting examples of aromatic solvents are benzene, xylene, nitro benzene, and toluene. In further embodiments, the solvent 146 may comprise a mixture of a polar organic solvent and a non-polar organic solvent. Some examples of non-polar organic solvents are hexane, cyclohexane, pentadecane, petroleum ethers, and dodecane. In embodiments using mixed solvent systems, the carboxylate salts may be soluble in the polar solvent and the AR products may be soluble in the non-polar solvent, and thus these materials may be easily separated from the reactants.

In some embodiments, the solvent 146 used in the anolyte 116 may comprise an ionic liquid (IL). An non-limiting example of an IL is a phosphonium based cation with four substituents. In one embodiment, the four substituents of the phosphonium cation are each independently an alkyl group, a cylcoalkyl group, an alkenyl group and an aryl group. In another embodiment, some/all of the substituents are of a similar group. In still another embodiment, some/all of the substituents are the same compound. In some embodiments, the anion of the ionic liquid is a carboxylate ion, more preferably, a carboxylate ion that is similar to the alkyl carboxylate anion being oxidized during the electrolysis; or most preferably, the carboxylate ion that is the same alkyl anion being oxidized during the electrolysis.

Certain alkali ion conductive membranes 3, such as NaSICON and LiSICON-type membranes, have a high temperature tolerance and thus the anolyte solution 116 may be heated to a higher temperature (or vice versa) without substantially affecting the temperature of the catholyte solution 117 or the functionality of the membrane 3. This means molten salts or acids may be used (in some embodiments) as the solvent 146 to dissolve the carboxylate salts 130/130a in the anolyte 116. Thus, in one embodiment, the solvent 146 is the molten salt of the carboxylate anion that is being oxidized.

The anolyte solution 116 may optionally contain a supporting electrolyte which is soluble in the solvent and provides high electrolyte conductivity in the anolyte solution 116. Non-limiting examples of supporting electrolytes include an alkali metal hydroxide, alkali metal salts, tetrafluoroborate, tetramethylammonium hexafluorophosphate, tetrabutylammonium tetrafluorobotate, tetramethylammonium perchlorate, and tetraethylammonium perchlorate. It should be appreciable to those skilled in the art that other soluble ionic compounds may be used as a supporting electrolyte.

In one embodiment, the catholyte 117 may be comprised of water and an unsaturated alkali hydroxide. The hydroxide concentration is between 0.1 and 50% by weight, or more preferably, between 5 and 25% by weight, or most preferably, between 7 and 15% by weight. Another embodiment may be constructed in which the catholyte 117 comprises alkali methylate. The temperature of the catholyte 117 may or may not be the same temperature of the anolyte 116 (as described above).

When a potential is applied to the cathode 5, a reduction reaction occurs. When the catholyte solution 117 is an aqueous based solution, water may be reduced to hydrogen gas 23 and hydroxide ions. The hydroxide formed can then combine with the alkali ion that is transported through the ion conducting membrane 3 to form an alkali hydroxide 140. This means that the alkali hydroxide (MOH 140) concentration of the catholyte 117 may increase as the electrolysis is performed.

The catholyte product stream may comprise a base which may be used to neutralize the carboxylic acid, thereby producing the alkali metal salt of the carboxylic acid. More specifically, the alkali metal salts of the carboxylic acids 130/130a may be formed (prior to having these materials enter the anolyte 116) by reacting the carboxylic acids with a base (such as NaOH, NaOR, KOH, LiOH, LiOR, KOR, etc.) These reactions are shown below:

R₁CO₂M+MOH→R₁CO₂M+H₂O

R₁CO₂M+MOR→R₁CO₂M+ROH

R₂CO₂M+MOH→R₂CO₂M+H₂O

R₂CO₂M+MOR→R₂CO₂M+ROH

As noted above, the cathode compartment 2 may regenerate the base as part of the reaction (e.g., producing MOH or MOR). Thus the cell 100 may regenerate the base (MOH or MOR) that was consumed by the acid neutralization step. Accordingly, the regenerated base may be recovered and re-used in future acid neutralization reactions or other chemical processes.

When an electrical potential is applied to the anode 4, oxidation occurs. In one embodiment, the oxidation of carboxylic acids or carboxylate anions leads to decarboxylation, producing carbon dioxide and carboxylate radicals. This decarboxylation reaction is shown below:

R₁CO₂M→R₁.+CO₂+M⁺+1e⁻

R₂CO₂M→R₂.+CO₂+M⁺+1e⁻

The radicals formed during this decarboxylation can then combine with similar radicals to form homocoupling products, or a radical of a different carboxylate and form heterocoupling products. These reactions are shown below:

R₁.+R₁.→R₁→R₁ (homocoupled product)

R₂.+R₂.→R₂→R₂ (homocoupled product)

R₁.+R₂.→R₁—R₂ (heterocoupled product)

In cases where both aliphatic and aromatic carboxylic acids or carboxylate anions are oxidized, the heterocoupling can form alkyl-aryl compounds. In another embodiment, the oxidation produces a radical or carbocation of an alkyl carboxylic acid or anion. The radical or carbocation can then participate in electrophilic substitution reactions with any aromatic groups that are present forming alkyl-aryl compounds.

Thus, as shown in FIG. 1, the products that may be obtained from the anode compartment 2 are carbon dioxide 25 along with R₁—R₁ 24a, R₂—R₂ 24b and/or R₁—R₂ 24c.

For example, if an alkali benzoate (C₆H₅CO₂M) were used as one of the acid salts of carboxylic acids, then this compound may decarboxylate to form phenyl radicals (C₆H₅.). Likewise, if the anolyte 116 also contained an alkyl carboxylate (R₁CO₂M), then this material would decarboxylate to R₁ radicals (R₁.). These R₁ radicals (R₁.) could then combine with the phenyl radicals (C₆H₅.), thereby forming C₆H₅—R₁, which is an alkyl-aryl compound. In other words, the radical reactions are operated to produce an AR compound via alkyl-aryl coupling. The above-recited example may be similarly applied to other aromatic acid salts—e.g., aromatic compounds that will produce radicals when decarboxylated).

In one embodiment, the electrolytic cell 100 may be operated in a continuous mode. In continuous mode, the cell 100 is initially filled with anolyte solution 116 and catholyte solution 117 and then, during operation, additional solution 116, 117 is fed into the cell 100, and products, by-products, and/or diluted solutions are removed from the cell 100 without ceasing operation of the cell 100. In another embodiment, the electrolytic cell 100 is operated in batch mode. In batch mode, the anolyte solution 116 and catholyte solution 117 are fed initially into the cell 100 and then the cell 100 is operated until a desired concentration of the product is produced, then the cell 100 is emptied and the products are collected. The cell 100 is then refilled to start the process again. Also, in either method, the feeding of solutions 116, 117 into the cell 100 may be done using a pre-made solutions or using components that form the solutions in situ. It should be noted in both continuous and batch mode, the anolyte 116 can be added to maintain the alkali ion concentration at a certain level.

As disclosed above, the anolyte solution 116 may comprise a solvent 146, and at least one alkali metal salt of a carboxylic acid 130/130a. The choice of the first carboxylic acid 130 is dependent on the desired structure of the alkyl component of the AR compound being synthesized. These will have a general formula of R₁CO₂M, where R₁ is a hydrocarbon with a carbon number from 2 to 22. Some non-limiting examples are, butyric acid, lactic acid, 3-hydroxypropanoic acid, valeric acid, myristic acid, palmitic acid, stearic acid, lauric acid, oleic acid, levelunic acid, naphthenic acid, etc. The carboxylic acid can also have functional groups present although the majority of the alkyl component should contain single carbon-carbon bonds. In one embodiment, the anolyte 116 can contain a mixture of alkyl based carboxylate salts.

The anolyte 116 may contain at least one carboxylate salt of a second type of carboxylic acid 130a. This carboxylic acid 130a will have the general formula of R₂CO₂H, where R₂ is an aromatic substituent such as a benzene or naphthalene ring. Some non-limiting examples are benzoic acid, naphthoic acid, naphthalenedicarboxylic acid, pamoic acid, hydroxynaphthoic acid, phenylpropanoic acid, phenylbutanoic acid, phenylethonic acid, naphthoic acid, phthalic acid, and trimesic acid. The aromatic carboxylic acid can also have additional functional groups and/or have multiple aromatic systems. In one embodiment, the anolyte 116 can contain a mixture of aryl based carboxylate salts.

As already disclosed, the anolyte solution 116 may comprise an aromatic compound that is used as the solvent 146. This aromatic solvent may contain an alkyl carboxylic acid or an alkali metal salt of an alkyl carboxylic acid or a mixture thereof. In some embodiments, the aromatic solvent contains a supporting electrolyte. In other embodiments, the anolyte solution 116 may comprise a mixture of an aromatic solvent and a polar organic solvent (that in combination form the solvent 146), where this solvent mixture contains at least one alkali metal salt of a carboxylic acid.

The R₁CO₂M and R₂CO₂M salts 130, 130a may be added to a suitable electrolyte which is used as the anolyte solution 116. Depending on the desired product, the anolyte 116 can contain a mixture of more than two types of carboxylic acids. The anolyte solution 116 may optionally include a supporting electrolyte if the conductivity of the anolyte 116 is not optimized for the decarboxylation. The anolyte solution 116 may be fed either continuously or in batch mode into the electrochemical cell, such as cell 100 shown in FIG. 1.

The applied electric potential 11 causes a reaction to occur at the anode 4. This reaction causes the decarboxylation of the carboxylate ions leading to the formation of carbon dioxide, and radicals of (R.) according to the reactions shown above. Depending on the conditions which the electrolysis is carried out, the radicals formed in the decarboxylation step can undergo the different coupling reactions (e.g., heterocoupling or homocoupling), as shown above.

The decarboxylation will permit the homocoupling of the alkyl carboxylate radicals, homocoupling of the aryl carboxylate radicals, and heterocoupling between the two types of radicals. The conditions and parameters used in the present embodiments can be modified to promote the heterocoupling over the homocoupling, and vice versa. The conditions and parameters can also be used to cause multiple alkylations onto a single aryl group. The products obtained from the coupling reactions can then be separated as needed to obtain a material that has the properties required for the lubricant or lubricant additive (or another desired product).

Additional embodiments may be designed in which the applied electrical potential 11 causes the oxidation at the anode 4 to decarboxylate an alkali salt of an alkyl carboxylic acid 130 or mixture of alkyl carboxylic acids 130, 130a in the presence of an aromatic solvent 146. (Of course, the aromatic solvent may be a solvent mixture containing an aromatic compound.) In this embodiment, the decarboxylation can lead to the formation of a radical as describe in the previous embodiment, or it can lead to the formation of a carbocation as shown in the following reaction.

R₁CO₂M→R₁⁺+CO₂+M⁺+2e⁻

In this embodiment the radical and/or the carbocation can act as an electrophile and subsequently be involved in an electrophilic substitution reaction. In such a reaction, the electrophile substitutes one of the substituents on an aromatic group, for example, hydrogen, as shown below as a non-limiting example.

R₁⁺+C₆H₆→C₆H₅—R₁+H⁺

R₁.+C₆H₆→C₆H₅—R₁+H.

(The H⁺ ions or the H. may then be consumed, further reacted, etc., in the chamber.) In the embodiment shown above, benzene is shown as the aromatic solvent. Those skilled in the art will appreciate that other aromatic organic solvents may also be used.

According the embodiments described herein, the product of the initial alkyl-aryl coupling reactions will have an aromatic group still present. In both cases (e.g, whether this occurs through heterocoupling or through a reaction with an aromatic solvent), this aromatic group can then go through further electrophilic substitution reactions with additional radicals or carbocations that are generated at the anode 4. By controlling of the conditions and parameters of the electrolysis, one can control the degree of alkylation that occurs on the aromatic group and thus control whether MAR products, DAR products, or PAR products are obtained.

The oxidation reaction causing the decarboxylation which forms the radical is usually conducted at high current density. To allow the high current density to occur with low voltages, a highly conductive catholyte 117 may be used in the cathode compartment 2 of the cell 100. Non-limiting examples of such catholyte materials 117 are aqueous alkali hydroxide and non-aqueous methanol/alkali methoxide solutions. As the potential across the cell 100 permits oxidation to occur at the anode 4, the potential also causes the reduction of the catholyte 117 to occur at the cathode 5. (This reduction reaction leads to the formation of hydrogen gas 23 and alkali metal hydroxides.)

Some advantages of this embodiment, using the alkali metal salt of the alkyl carboxylic acid instead of the carboxylic acid itself are:
RCO₂M is more polar than RCO₂H and so more probable to decarboxylate at lower voltages;
The electrolyte conductivity may be higher for alkali metal salts than the acid solutions; and
The anolyte and catholyte solution can be completely different, allowing favorable reactions to take place at either/both electrodes.

The following examples are given to illustrate various embodiments within the scope of the present disclosure.

### EXAMPLES

Several examples will be given to demonstrate the technical feasibility of coupling alkyl and aryl compounds to form compounds with properties that are beneficial for lubricants (or other similar compounds), using the electrochemical decarboxylation process at low temperatures and pressures. The examples demonstrate the decarboxylation of mixtures of sodium salts of carboxylic acids either consisting of mainly aliphatic or mainly aromatic components, using electrolytic cells equipped with a NaSelect® NaSICON membrane manufactured by Ceramatec, Inc., Salt Lake City, Utah. The decarboxylation produces alkyl-aryl compounds, which may be used in the production of Group V lubricants or other compounds.

The examples disclosed herein, used an experimental setup which is schematically shown in FIG. 1. The cell employed for these experiments was a micro flow cell, allowing both the anolyte and catholyte to be pumped through the cell while minimizing the distance between the electrodes and the membrane. The membranes used in the examples consisted of 2.54 cm diameter NaSICON disks of about 1 mm thickness which were housed on scaffolds in the center of the cells. As the scaffold and membrane physically separate the anode and cathode compartments, there was a separate reservoir and temperature controlled hotplate for the anolyte and catholyte. This allowed the chemistry and conditions of each electrolyte to be optimized for the respective electrode reactions. A multiple-head parasitic pump was used to pump both electrolytes into the electrolysis cell, and, depending on the temperature of the electrolytes, the tubing between the cell, pump, and reservoir was insulated.

The anolyte, which contains the sodium salts of the carboxylic acids, is made by dissolving at least 10% by weight of both salts into a polar organic solvent. This was conducted using two methods. For the first method, the sodium salts were prepared directly in the polar organic solvent by the addition of the carboxylic acids and NaOH. To ensure the complete de-protonation of the acid, the cell was operated at a pH between 8 and 12, indicative of excess NaOH. The second method consisted of preparing the sodium salts in separate solutions following conventional saponification reactions and then dissolving the prepared salt into a polar organic solvent. For this method, a general saponification procedure was used during which the sodium carboxylates form as the carboxylic acid is neutralized. In this case, the carboxylic acids were converted to the sodium salts separately and then added to the polar solvent. The catholyte can be made from any solution containing sodium salts, and for the examples given herein, an aqueous sodium hydroxide solution was used. To obtain low solution resistance, the temperatures of the electrolytes were increased to 50° C. to improve both the solubility and conductivity.

Once the reservoirs reached the desired temperatures, a power supply (BP Precision 1786B) was connected and a current density between 10 and 100 mA/cm² was applied. During the electrolysis, the voltage and current were monitored using a Data Acquisition Unit (Agilent 3490A) controlled by LabVIEW. The applied current density caused oxidation to occur at the anode (smooth platinum) and reduction to occur at the cathode (nickel), with each electrode having an area of 11 cm². As the power supply transports electrons from the anode to the cathode, a charge balance must be maintained across the cell by the diffusion of positively charge ions. Given the high selectivity of the NaSICON membrane for Na⁺ ions, it is the only species that can provide this balance, thus a high concentration of the sodium salt was desired.

Methanol was the solvent used in the examples given. In methanol, the solubility of the all the sodium salts used in the examples were found to be 10-15% by weight after the addition of mild heat. In all examples, when both salts are present, the solubility of each salt in methanol was found to be 10% by weight. (Thus, the solutions containing two salts had a total salt concentration of 20% by weight.)

Analysis of the products formed in this system was performed using gas chromatography (GC) and mass spectroscopy (MS). The presence of sodium salts made the direct analysis of the GC-MS analysis of the products not possible. Therefore, different fractions were obtained at different stages of the post processing procedure and were analyzed with GC-MS. The post processing consisted of different steps involving solvent extraction, physical separation and distillation. The specific processing used for the examples given is discussed below. GC analysis of these systems was performed using a 15 meter metal column with low polarity crossbond diphenyl dimethyl polysiloxane phase which can handle temperatures between −60 and 430° C. A temperature program was used that held the temperature 40° C. for 5 minutes then increased the temperature at 10°/minute to 320° C. Once the temperature was 320° C., this temperature was held for 10 minutes. The GC-MS analysis was conducted using a 60 meter column with a non-polar dimethylpolysiloxane phase which can handle a temperature range between −60 and 325° C. A temperature program was used that started the temperature at 35° C. and increased the temperature to 310° C. at 10°/minute and then held this temperature for 35 minute. The mass spec range used to analyze the data was 29 to 550 m/z.

### Example 1

The electrochemical decarboxylation process disclosed herein was used to alkylate an aromatic ring with a long chain aliphatic group. The prepared alkylated aromatic compound may have properties that are beneficial for components of Group V lubricants. The anolyte for this example consisted of 10% by weight sodium oleate and sodium benzoate in methanol. To prepare the anolyte, the sodium salts of benzoic and oleic acid had to be prepared from the corresponding acids. This was preformed individually by adding the acids at 20% by weight to methanol and heating the solution to 50° C. To the heated solution, 7% by weight sodium hydroxide was added, upon which white solids crashed out. After removing the methanol from the solids, the solids were allowed to dry overnight. The anolyte was prepared by adding 10% by weight sodium benzoate and 10% by weight sodium oleate to methanol. An aqueous solution containing 10% by weight sodium hydroxide was used as the catholyte.

The electrolysis was conducted in batch mode, during which the anolyte and catholyte were cycled through the corresponding anode and cathode compartments of the cell. Both electrolytes were maintained at a temperature of 50° C. A current density of 18 mA/cm² was applied to the cell until enough charge passed to theoretically convert 40% of the total sodium salt. The reactions that occurred during the electrolysis in the anode compartment are shown below.

CH₃(CH₂)₁₆CO₂Na→CH₃(CH₂)₁₅CH₂.+CO₂+Na⁺+1e⁻

C₆H₅CO₂Na→C₆H₅.+CO₂+Na⁺+1e⁻

The reaction shown below simultaneously occurred in the cathode compartment:

2H₂O+2e⁻→2OH⁻+H₂

The CH₃(CH₂)₁₅CH₂. species is an alkyl radical derived from sodium oleate. The C₆H₅. is a phenyl radical derived from the sodium benzoate.

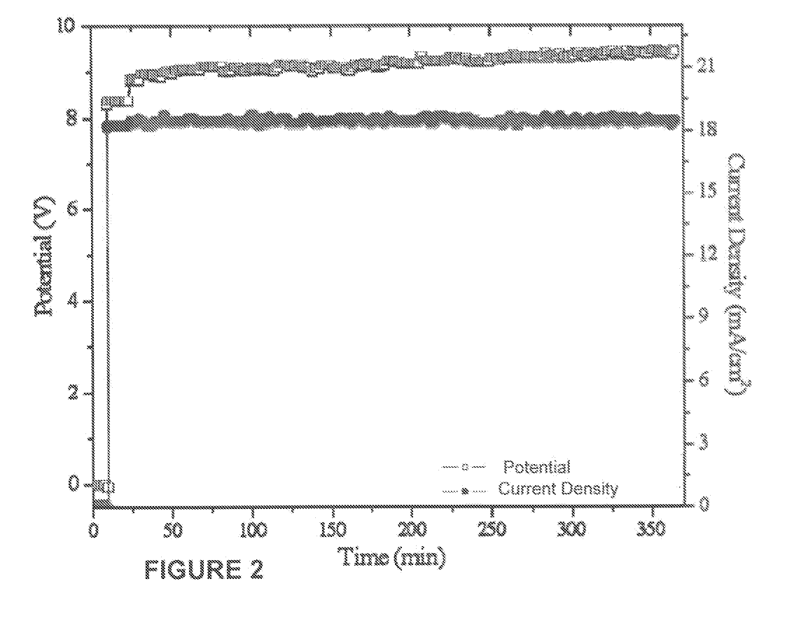

The decarboxylation which occurred in the anode compartment produced CO₂. The CO₂ was bubbled through the calcium hydroxide solution forming calcium carbonate. The calcium carbonate was then analyzed using TGA. FIG. 2 contains a graph showing voltage and current density verses time for this example, which shows that a potential of 9 V was produced at the applied current density. The cell held this constant voltage for the duration of the 6 hour experiment. The experiment was terminated at this point, and the anolyte was processed so it could be analyzed with GC. The condition used in this example promoted both homo- and hetero-coupling of the radicals formed at the anode, permitting the following coupling reactions to occur.

2CH₃(CH₂)₁₅CH₂.→CH₃(CH₂)₃₂CH₃ (homocoupling of alkyl radicals)

2C₆H₅.→C₆H₅.C₆H₅ (homocoupling of aromatic radicals)

C₆H₅.+CH₃(CH₂)₁₅CH₂.C₆H₅—(CH₂)₁₆CH₃ (heterocoupling of aromatic radical with alkyl radicals)

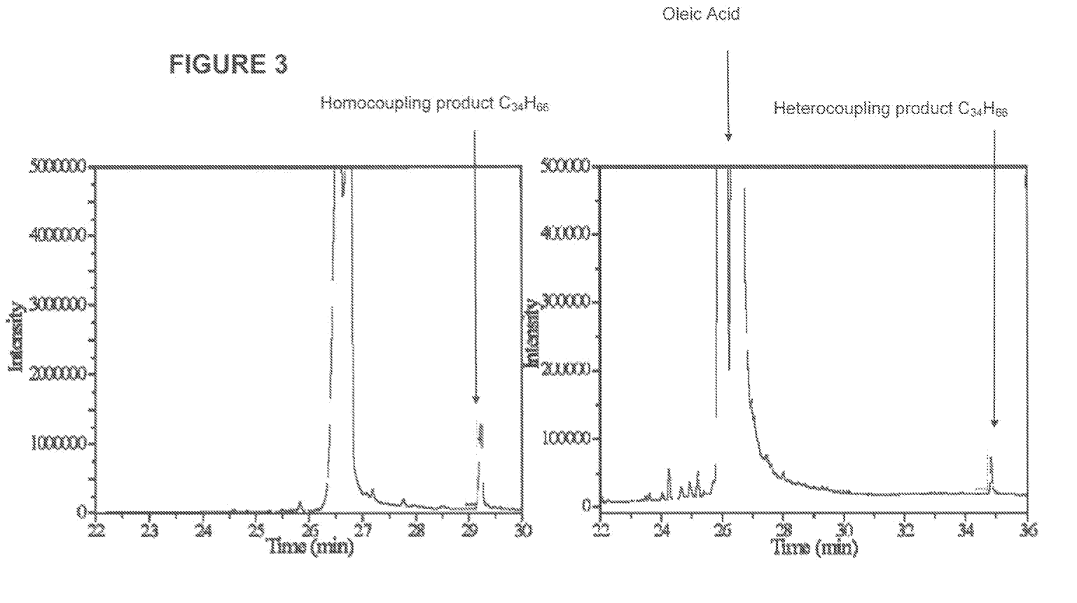

After the termination of the electrolysis, the anolyte was processed so it could be analyzed with GC. The processing consisted of adding 30% H₂SO₄ to the anolyte solution which caused Na₂SO₄ to crash out. After centrifuging, the methanol solution was decanted off of the solid material. The liquid was then mixed with ethyl acetate. Using a separatory funnel, the layers were separated and the ethyl acetate layer was analyzed with GC. FIG. 3 shows a GC of the decarboxylation of a solution containing only sodium oleate. This GC shows the elution of the oleic acid at 26.5 minutes and the elution of the homocoupling product (from oleate) as 29.2 minutes. The second GC shown in FIG. 3 shows the analysis of the decarboxylation of sodium oleate and sodium benzoate. This GC shows the elution of the oleic acid at 26.5 minutes and the elution of the hetero-coupling alky-aryl compound at 34.9 minutes.

### Example 2

The electrochemical decarboxylation process disclosed herein was used to perform homo/hetero coupling on a mixture of carboxylic acids which contain straight chain aliphatic and aromatic groups, known as naphthenic acid. The prepared liquid may have properties that are beneficial for components of Group V lubricants. The anolyte for this example consists of 10% by weight sodium naphthenate in methanol. An aqueous solution containing 10% by weight sodium hydroxide was used as the catholyte.

The electrolysis was conducted in batch mode, during which the anolyte and catholyte were cycled through the corresponding anode and cathode compartments of the cell. Both electrolytes were maintained at a temperature of 50° C. The cell was started at a current density of 9 mA/cm², and current was applied to the cell until enough charge passed to theoretically convert 40% of the total sodium salt. The reactions that occurred during the electrolysis in the anode compartment are shown below.

RₙₐₚCO₂Na→Rₙₐₚ.+CO₂+Na⁺+1e⁻

In the above reaction Rₙₐₚ represents a mixture of cyclopentyl and cyclohexyl aliphatic groups with 9 to 20 carbon backbone and molecular weights between 120 and 700 amu. The reaction shown below is simultaneously occurring in the cathode compartment.

2H₂O+2e⁻→2OH⁻+H₂

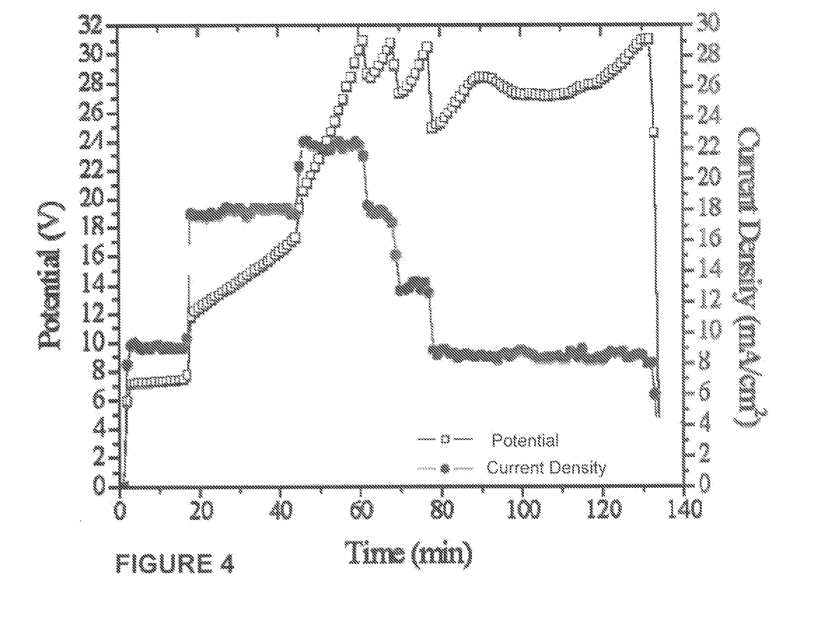

The decarboxylation occurring in the anode compartment produces CO₂ which is bubbled through a calcium hydroxide solution forming calcium carbonate. The calcium carbonate was then analyzed using TGA. FIG. 4 contains a graph showing voltage and current density verses time for this example. The experiment was started by applying a current density of 9 mA/cm² and then this current density was increased stepwise to 22 mA/cm², at which time the cell potential reached 31 V. This limit was reached within the first hour, so the current density was decreased to 19 mA/cm² until the potential reached the 31 V limit, which occurred within 1 hour. As shown in FIG. 4 the current density was then stepped down three more times before the experiment was terminated after 2 hours. The conditions used in this example promoted coupling of the mixture of radicals formed at the anode. Accordingly, this reaction formed a mixture of compounds with the general structures shown below, as a non-limiting example.

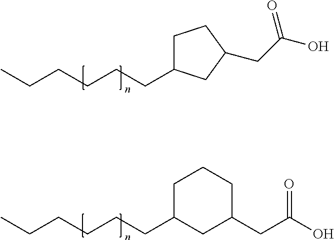

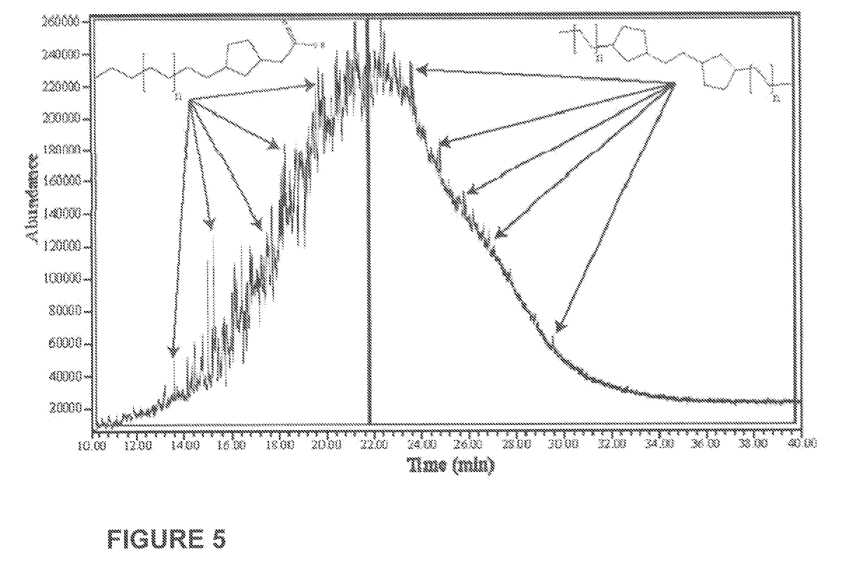

After the termination of the electrolysis, the anolyte was processed so it could be analyzed with GC. The post reaction processing consisted of adding 30% H₂SO₄ to the anolyte solution which caused Na₂SO₄ to crash out. A viscous liquid also formed after the addition of H₂SO₄, and this liquid was used for GC-MS analysis. The GC results of this liquid are shown in FIG. 5. This figure shows that a broad peak was obtained, indicative of a mixture of compounds with slight differences in structure and/or mass. Using MS, the identity of the species of this mixture was determined to be the combination of the starting carboxylic acids and a mixture of the coupled products produced by the decarboxylation of these acids.

### Example 3

Another example of the present embodiments incorporates the alkyl-aryl coupling of EXAMPLE 1 with the coupling demonstrated in EXAMPLE 2. In this example, the electrochemical decarboxylation process leads to the coupling of a mixture of long chain aliphatic radicals containing rings to be coupled with naphthoate radicals. The prepared alkylated naphthalene may have properties that are beneficial for components of Group V lubricants. The anolyte for this example consists of 10% by weight sodium naphthenate and sodium naphthoate in methanol. To prepare the anolyte, 10% by weight naphthoic acid was added to methanol followed by the addition of 4% by weight sodium hydroxide. To this solution, 10% by weight sodium naphthenate was added. An aqueous solution containing 10% by weight sodium hydroxide was used as the catholyte.

The electrolysis was conducted in batch mode, during which the anolyte and catholyte were cycled through the corresponding anode and cathode compartments of the cell. Both electrolytes were maintained at a temperature of 50° C. A current density of 27 mA/cm² was applied to the cell until enough charge passed to theoretically convert 40% of the total sodium salt. The reactions that occurred during the electrolysis in the anode compartment are shown below.

RₙₐₚCO₂Na→RₙₐₚCH2+CO₂+Na⁺+1e⁻

C₁₀H₇CO₂Na→C₁₀H₇.+CO₂+Na⁺+1e⁻

Where Rₙₐₚ represents the mixture of carboxylic acids that make up what is termed naphthenic acid. The reaction shown below is simultaneously occurring in the cathode compartment.

2H₂O+2e⁻→2OH⁻+H₂

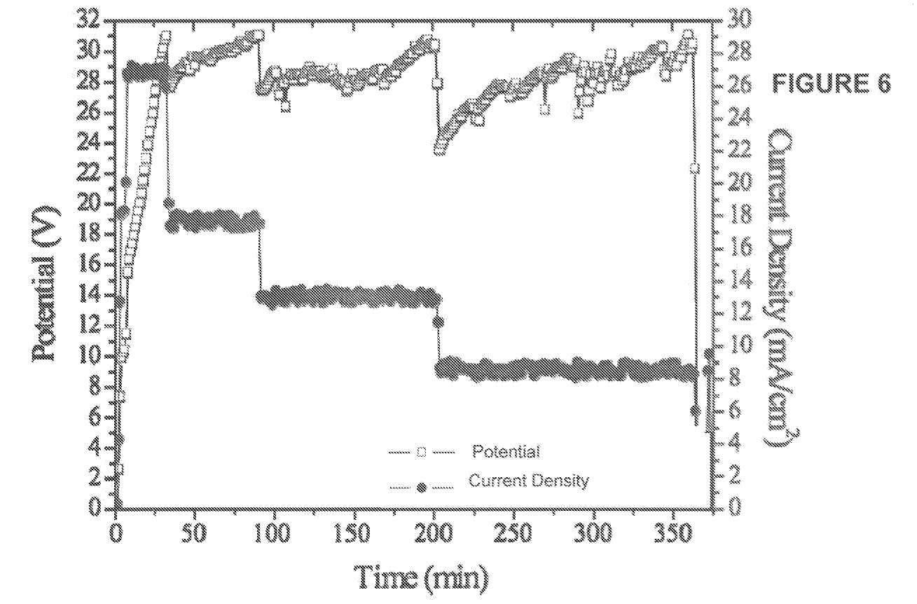

The decarboxylation occurring in the anode compartment produces CO₂ which is bubbled through the calcium hydroxide solution forming calcium carbonate. The calcium carbonate was then analyzed using TGA. FIG. 6 contains a graph showing voltage and current density verses time for this example. The experiment was started by applying a current density of 27 mA/cm² until the voltage of the cell reached 31V. This limit was reached within the first hour, so the current density was decreased to 19 mA/cm² until the potential reached the 31 V limit, which occurred within another 1 hour period. As shown in FIG. 6, the current density was then stepped down two more times before the experiment was terminated after 6 hours. The conditions used in this example promoted both homo- and hetero-coupling of the radicals formed at the anode, permitting the following reactions to occur.

2Rₙₐₚ.→Rₙₐₚ—Rₙₐₚ (homocoupling of radicals)

2C₁₀H₇.→C₁₀H₇—C₁₀H₇ (homocoupling of radicals)

C₁₀H₇.+Rₙₐₚ.→C₁₀H₇—Rₙₐₚ (heterocoupling of aromatic radical with alkyl radical)

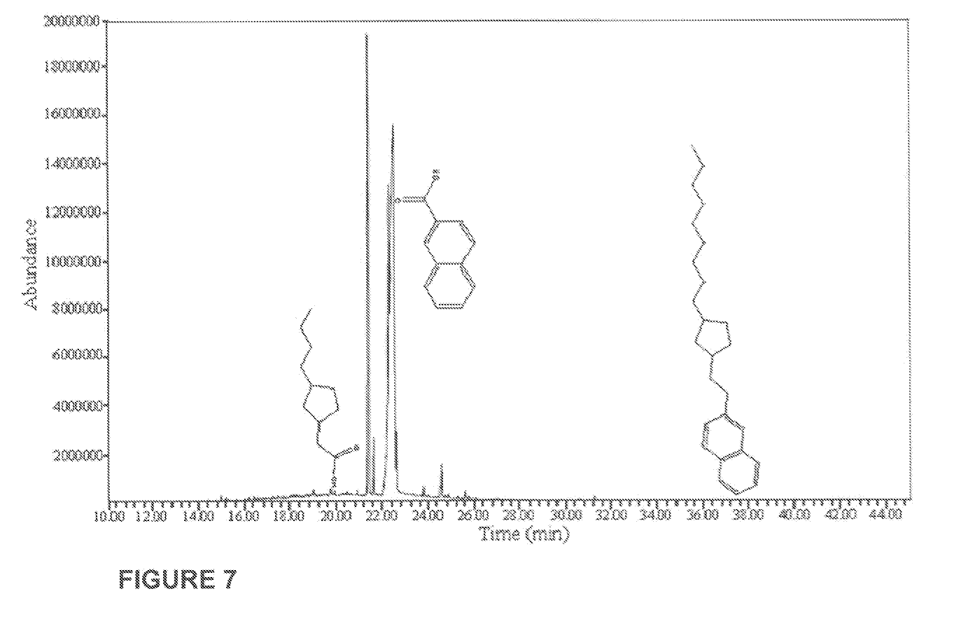

After the termination of the electrolysis, the anolyte was processed so it could be analyzed with GC. The processing consisted of adding 30% H₂SO₄ to the anolyte solution which caused Na₂SO₄ to crash out and a viscous liquid to form. After centrifuging, the methanol layer was separated from the solid and viscous liquid, and then the methanol layer was completely distilled leaving a new solid and a liquid. This new solid and new liquid did not boil under vacuum and temperatures up to 180° C. The solid was filtered from the liquid and was dissolved in ethyl acetate for the GC-MS analysis. The results from this solid in the ethyl acetate are shown in FIG. 7. The structures shown on the GC in FIG. 7 were determined using MS. The MS shows that this fraction of the reaction solution contained the starting naphthenic and naphthoic acids and a small peak due to the elution of the heterocoupling product.

### Example 4

An example of another embodiment will be given, where the same carboxylic acids used in EXAMPLE 3 were used to perform alkyl-aryl coupling. In this example, the anolyte consisted of a non-homogenous mixture of a polar electrolyte and a non-polar electrolyte. The polar electrolyte was methanol and the non-polar electrolyte was pentadecane. In the methane portion of the electrolyte, 10% by weight sodium naphthenate and sodium naphthoate, were dissolved. This was prepared following the method described in EXAMPLE 3, and then pentadecane was added to this layer at 20% by volume.

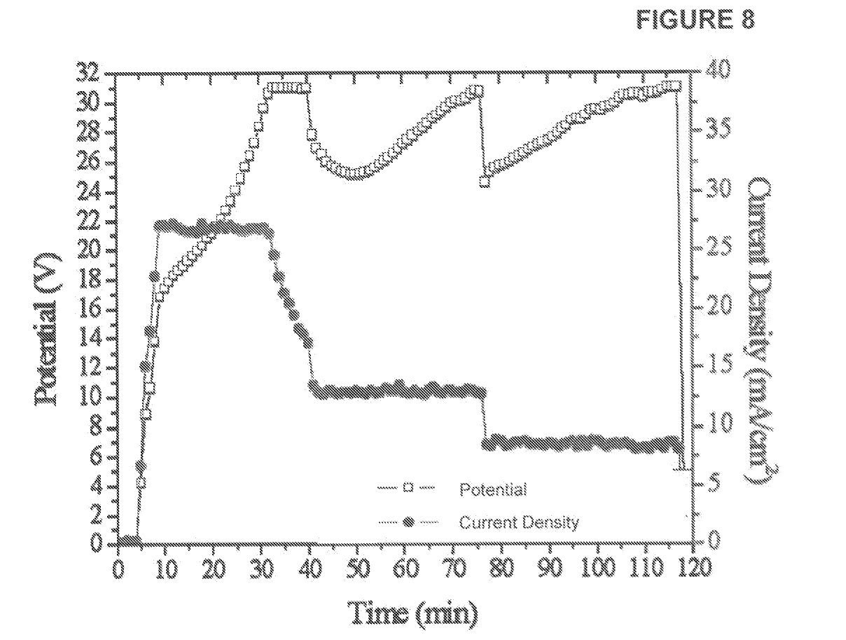

The electrolysis was conducted in batch mode, during which the anolyte and catholyte were cycled through the corresponding anode and cathode compartments of the cell. The anolyte compartment was setup so that the anolyte was pulled into the cell from the methanol layer and returned to the cell in the pentadecane layer. This was chosen to help selectively pull the polar solvent into the cell for electrolysis, and allow the product obtained to be exchanged into the non-polar solvent following the electrolysis. Both electrolytes were maintained at a temperature of 50° C. FIG. 8 contains a graph showing voltage and current density verses time for this example. A current density of 27 mA/cm² was applied to the cell until the potential reached 31 V, and then the current density was stepped down two more times before the experiment was terminated at 2 hours. The reactions that occurred during the electrolysis in the anode compartment are shown below.

RₙₐₚCO₂Na→RₙₐₚCH2+CO₂+Na⁺+1e⁻

C₁₀H₇CO₂Na→C₁₀H₇.+CO₂+Na⁺+1e⁻

Where Rₙₐₚ represents the mixture of carboxylic acids that make up what is termed naphthenic acid. The reaction shown below is simultaneously occurring in the cathode compartment.

2H₂O+2e⁻¹→2OH⁻+H₂

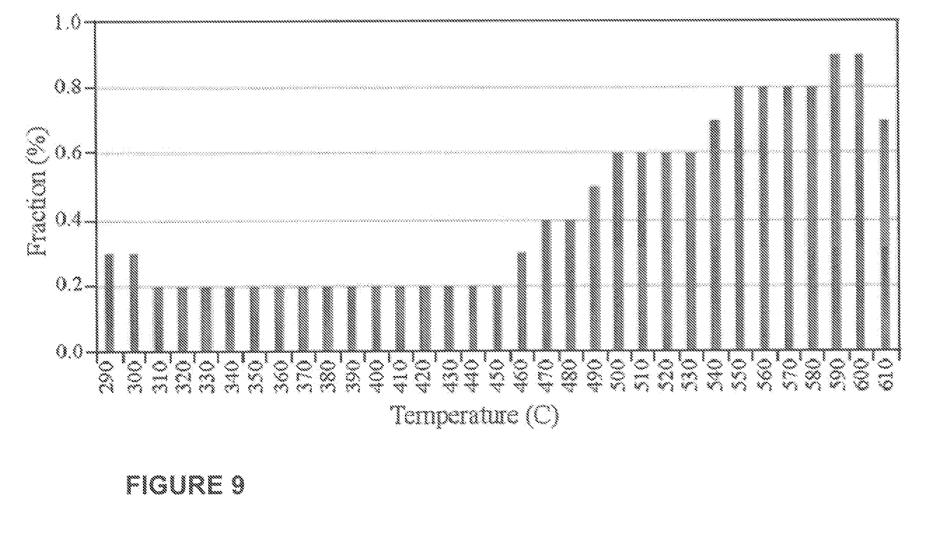

After termination of the electrolysis, the pentadecane and the methanol were separated using a separatory funnel. The pentadecane layer was then analyzed using a GC setup to run a SimDist (simulated distillation). The results of this analysis are shown in FIG. 9, which shows that the alkyl-aryl coupling products that partitioned into the pentadecane had the desired thermal properties for a Group V lubricant.

### Production of Styrene and Styrene Precursors

Styrene is a desirable chemical in that is it used to make many polymeric materials and plastic materials, including ABS (Acrylonitrile butadiene styrene). Styrene has the formula C₆H₅CH₂CH₂. It has the chemical structure of:

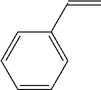

Currently about 15 billion pounds of styrene are produced annually, mostly from the catalytic dehydrogenation of ethylbenzene. This process requires high temperatures and pressures and can often produce styrene with impurities such as sulfur which may operate to inhibit polymerization reactions. However, the present decarboxylation methods, as outlined herein, provides a ready method for preparing styrene.

Specifically, if an alkali metal benzoate (from benzoic acid) and an alkali metal lactate (from lactic acid (2-hydropropanoic acid) or 3-hydroxypropanoic acid) are decarboxylated in the same cell, then a styrene precursor could be readily made. (The hydropropanoates and benzoate are listed above as being moieties that could be used as part of the present alkyl-aryl coupling reactions.) A lactate (2-hydropropanoic acid) anion (CH₃CH(OH)CO₂⁻) has the following formula.

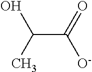

The 3-propanoic acid anion (CH₂(OH)CH₂CO₂⁻) has the following formula

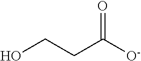

The alkali metal benzoate (from benzoic acid) may be decarboyxlated as follows:

C₆H₅CO₂Na→C₆H₅.+CO₂+Na⁺+1e⁻

Likewise, an alkali metal lactate and/or 3-hydroxpropanoate (from lactic acid or 3-hydroxypropranoic acid) may be decarboxylated as follows:

CH₃CH(OH)CO₂Na→CH₃CH(OH).+CO₂+Na⁺+1e⁻

(The alkali metal benzoate and the alkali metal lactate may be formed from benzoic acid and lactic acid, using a saponification reaction as outlined herein.)

If these alkali metal salts are mixed in the same anolyte and decarboxylated in the same cell, the following heterocoupling reaction (which involves the coupling of an alkyl group and an aromatic group) may occur:

C₆H₅.+CH₃CH(OH).→C₆H₅—CH(OH)CH₃

The produced product is 1-phenylethanol. In the case when the decarboxylcation is performed in the presence of 3-hydroxypropanoate, then 2-phenylethanol is the product. (As noted above, the particular conditions of the cell may be selected to foster heterocoupling as opposed to homocoupling. However, some of the homocoupled radical products (C₆H₅—C₆H₅ and CH₃CH(OH)—CH(OH)CH₃) may also be obtained and separated out from the phenylethanol. (CH₃CH(OH)—CH(OH)CH₃ is 2,3-butanediol.) It should also be noted that, in additional or in an alternative, to 1-phenylethanol and 2,3-butanediol, quantities of 2-phenylethanol and 1,4-butanediol may also be produced (due to different reaction mechanisms/pathways.)

In one embodiment, the homocoupling product (C₆H₅—C₆H₅) may be obtained and separated out from the 1-phenylethanol and the 2,3-butanediol (or 2-phenylethanol and 1,4-butanediol). This separation could leave a mixture of 1-phenylethanol and 2,3-butanediol (or 2-phenylethanol and 1,4-butanediol), which then may be subjected to a dehydration reaction to produce both styrene and butadiene, both are components used to make ABS polymeric materials. (Butadiene has a formular of CH₂CHCHCH₂.) The butadiene and the styrene could then be used to make ABS or other thermoplastics, as desired.

In one embodiment, the decarboxylation of the hydroxypropanoic acid and benzoic acid and/or salts of the acids can be performed in an electrolyte that uses acrylonitrile as the solvent. (The electrolyte could also be a mixture of solvents that contains acrylonitrile.) In such an embodiment, all three components needed to make ABS materials would be present. In another embodiment, the decarboxylation of sodium lactate or sodium 3-hydroxpropanoate can be performed in an electrolyte that contains benzene or a mixture of benzene and a polar organic solvent. In this embodiment, the radical or carbocation can then undergo electrophilic substituent reaction with the benzene and form the 1-phenylethanol, or the 2-phenylethanol.

Once the phenylethanol (e.g., the 1-phenylethanol or the 2-phenylethanol) is obtained, this product may be subjected to a dehydration reaction to produce styrene.

C₆H₅—CH(OH)CH₃—C₆H₅—CH₂CH₂+H₂O

Likewise if 2,3-butantediol (or 1,4-butanediol) is present it may be subjected to a dehydration reaction to produce butadiene.

CH₃CH(OH)CH(OH)CH₃→CH₂CHCHCH₂+H₂O

Those skilled in the art will appreciate the conditions necessary to implement a dehydration reaction. One or more catalysts may be used to facilitate/speed this dehydration reaction. In some embodiments, there are a variety of different reaction conditions, catalysts, etc., that can be used to dehydrate 1-phenylethanol (or 2-phenylethanol) into styrene.

The above-recited method for producing styrene may not require high temperatures and/or high pressures, nor does it require an ethylbenzene precursor. Rather, the precursors are hydroxypropanoic acids and benzoic acids, which are readily available substrates. Likewise, there will likely be little or no sulfur impurities in the styrene, as are found in some styrenes made via other manufacturing methods. Thus, the resulting styrene may be purer than other commercially available styrene monomers. Also, the present method will likely provide a pathway to produce the both styrene and butadiene in one step, and possibly be produced in the presence of acrylonitrile.

All the patent applications and patents listed herein are expressly incorporated herein by reference.

What is claimed is:

1. An electrochemical cell comprising:
an anode compartment capable of housing a quantity of anolyte, the anolyte comprising a quantity of a first alkali metal salt of a carboxylic acid and an aromatic compound, wherein the first alkali metal salt of a carboxylic acid is an alkyl carboxylic acid;
an anode in communication with the anolyte;
a catholyte compartment capable of housing a quantity of catholyte;
a cathode in communication with the catholyte;
an alkali ion conducting membrane; and
a voltage source, wherein the voltage source decarboxylates the first alkali metal salt of the carboxylic acid and forms an aryl-alky coupled product.

2. The electrochemical cell of claim 1, wherein the voltage source decarboxylates the first alkali metal salt of the carboxylic acid into alkyl radicals, and wherein the aromatic compound comprises a second alkali metal salt of a carboxylic acid, wherein the voltage source decarboxylates the second alkali metal salt of a carboxylic acid into aryl radicals, wherein the aryl-alky coupled product is formed by coupling aryl radicals with alky radicals.

3. The electrochemical cell of claim 1, wherein the aromatic compound is a solvent, wherein the decarboxylation produces the aryl-alky coupled product via electrophillic substitution on an aromatic ring of the solvent.

4. The electrochemical cell of claim 1, wherein the aromatic compound comprises benzene.

5. The electrochemical cell as in claim 1, wherein the aryl-alky coupled product is a Group V lubricant.

6. The electrochemical cell as in claim 1, wherein the aryl-alky coupled product is 1-phenylethanol or 2-phenylethanol

7. The electrochemical cell as in claim 6, further comprising dehydrating the 1-phenylethanol or the 2-phenylethanol to form styrene.

8. The electrochemical cell as in claim 1, wherein the aromatic compound comprises a second alkali metal salt of a carboxylic acid, wherein the second alkali metal salt of a carboxylic acid is an alkali metal salt of one or more of the following acids: benzoic acid, phenylpropanoic acid, phenylbutanoic acid, phenylethonic acid, naphthoic acid, naphthoic acid, naphthalenedicarboxylic acid, pamoic acid, hydroxynaphthoic acid, phthalic acid, and trimesic acid.

9. The electrochemical cell as in claim 1, wherein the first alkali metal salt of a carboxylic acid is an alkali metal salt of one or more of the following acids: butyric acid, lactic acid, 3-hydroxypropanoic acid, valeric acid, myristic acid, palmitic acid, stearic acid, lauric acid, oleic acid, levelunic acid and naphthenic acid.

10. The electrochemical cell of claim 1, wherein the anolyte comprises:
a polar organic solvent or an ionic liquid;
a supporting electrolyte.

11. The electrochemical cell of claim 1, wherein the anolyte comprises a polar organic solvent mixed with a non-polar organic solvent.

12. The electrochemical cell of claim 1, wherein the ion conductive membrane is in the shape of a disk and is between is between 10 and 5000 microns thick, or preferably between 100 and 1000 microns thick, or even more preferably, between 200 and 700 microns thick.

13. The electrochemical cell of claim 1, wherein the ion conductive membrane is in the shape of a cylinder with a diameter between 0.25-25 cm, more preferably between 1.27-12.7 cm, or most preferably between 2.54-7.62 cm.

14. The electrochemical cell of claim 1, wherein the ion conductive membrane is in the form of disk with diameters between 0.25-25 cm, more preferably the diameter is between 1.27-12.7 cm, or most preferably between 2.54-7.62 cm and are assembled in a scaffold.

15. The electrochemical cell of claim 1, wherein by-products that are formed in addition to the aryl-alky coupled product comprise carbon dioxide and hydrogen gas.

16. The electrochemical cell of claim 1, wherein the aryl-alky coupled product is subjected to further electrophillic substitution.

17. The electrochemical cell of claim 1, wherein the wherein the aryl-alky coupled product is formed via coupling of an aryl radical with an alkyl radical, wherein the electrochemical cell also produces an aryl-aryl coupled product and an alkyl-alkyl coupled product.

18. A method for producing an aryl-alky coupled product comprising:
obtaining a first alkali metal salt of a carboxylic acid and an aromatic compound, wherein the first alkali metal salt of a carboxylic acid is an alkyl carboxylic acid;
decarboxylating the first alkali metal salt of the carboxylic acid into alkyl radicals, wherein the alkyl radicals react with the aromatic compound to produce an aryl-alky coupled product.

19. The method as in claim 18, wherein the first alkali metal salt of the carboxylic acid was formed via a saponification reaction using a base of the formula MOH or MOR, wherein, “M” represents an alkali metal and “OH” represents a hydroxide anion and “OR” represents an alkoxide anion.

20. The method of claim 19, wherein the base is re-formed as part of the decarboxylation, wherein the base is collected and re-used in a further saponification reaction.

21. The method of claim 18, wherein the aromatic compound comprises a second alkali metal salt of a carboxylic acid, wherein the second alkali metal salt of a carboxylic acid is decarboxylated into aryl radicals, wherein the aryl-alky coupled product is formed by coupling aryl radicals with alky radicals.

22. The method of claim 18, wherein the aryl-alkyl coupled product is 1-phenylethanol or 2-phenylethanol, wherein the method further comprises:
dehydrating the 1-phenylethanol or 2-phenylethanol into styrene; and
dehydrating 1,4-butanediol or 2,3-butanediol into butadiene.

23. The method of claim 18, wherein the aryl-alkyl coupled product is a Group V lubricant.

24. An electrochemical cell comprising:
an anolyte comprising a first alkali metal salt of a carboxylic acid and an aromatic compound, wherein the first alkali metal salt of a carboxylic acid is an alkyl carboxylic acid;
an anode in communication with the anolyte;
a catholyte;
a cathode in communication with the catholyte;
a voltage source, wherein the voltage source decarboxylates the first alkali metal salt of the carboxylic acid into alkyl radicals that react to form a aryl-alkyl coupled product.
